(19) United States
(12) Patent Application Publication
(10) Pub. No.: US 2009/0226884 A1
(43) Pub. Date: Sep. 10, 2009

(54) Method of Quantitative Analysis of Oxidized Protein, Labeling Reagents for Quantitative Analysis of Oxidized Protein and Labeling Reagent kit for Quantitative Analysis of Oxidized Protein

(75) Inventors: Kazuo Tsujimoto, Ishikawa-ken (JP); Akio Hayashi, Ishikawa-ken (JP); Takaaki Kawai, Kanagawa-ken (JP); Hiroyuki Matsumoto, Fukuoka-ken (JP)

Correspondence Address: KANESAKA BERNER AND PARTNERS LLP, 1700 DIAGONAL RD, SUITE 310, ALEXANDRIA, VA, 22314-2848, US

(73) Assignee: Japan Advanced Institute of Science and Technlogy, Ishikawa (JP)

(21) Appl. No.: 12/225,158

(22) PCT Filed: Mar. 20, 2007
(86) PCT No.: PCT/JP2007/055617

§ 371 Date: Nov. 13, 2008

(30) Foreign Application Priority Data
Mar. 20, 2006 (JP) 2006-077526

(51) Int. Cl. C12Q 1/00; G01N 33/68

(52) U.S. Cl. 435/4; 436/86

(57) ABSTRACT

Oxidized proteins, which have undergone oxidative modifications, are labeled with labeling reagents and quantified by mass spectrometry. In this process, a first labeling reagent, which is capable of reacting with the oxidized proteins, and a second labeling reagent, which has the same chemical structure as the first labeling reagent and in which at least part of the component atoms is substituted by an isotope of the atom concerned, are employed as the labeling reagents. The oxidized proteins labeled with the first labeling reagent and the oxidized proteins labeled with the second labeling reagent are mixed together and subjected to mass spectrometry, with mixing ratios varied. As the labeling reagents, are raised here 2,4-dinitrophenylhydrazine and 2,4-dinitrophenylhydrazine in which a carbon atom on the phenyl group has been substituted by a stable isotope (¹³C), for example.

### TECHNICAL FIELD

The present invention relates to a method of quantitative analysis of an oxidized protein, which is capable of quantitative analysis with high sensitivity a carbonylated protein that has undergone oxidative modifications, further to labeling reagents used in quantitative analysis of the oxidized protein and to a labeling reagent kit for quantitative analysis of the oxidized protein.

### BACKGROUND ART

Oxidation of proteins has been presumed to have something to do with arteriosclerosis, rheumatoid arthritis, emphysema, neurodegenerative diseases (such as Alzheimer disease, Parkinson's disease, etc.) and disorders including senescence, acute pancreatitis, cancer, etc. and, therefore, highly sensitive quantification of proteins that have undergone oxidative modifications has been needed. For example, there is the theory that low-density lipoprotein (LDL) denaturing by oxidation triggers arteriosclerosis. Thus, it is expected that active species inducing oxidation of the LDL is elucidated. In order to elucidate the active species, however, it is necessary to elucidate an oxidized site of the LDL and obtain data from the side of a product material.

The spectrometry has heretofore been adopted mainly as the technology for quantifying proteins and various quantifying methods have been studied. Since 2,4-dinitrophenylhydrazine (DNPH) forms a stable Schiff's base in cooperation with a carbonylated protein that has undergone oxidative modifications, for example, the measurement of the absorption of light having the ultraviolet wavelength thereof has been used for quantifying the carbonylated protein (refer, for example, to Methods Enzymol., 186: 464-478 (1990)). Furthermore, the two-dimensional electrophoresis is a method for developing a protein utilizing differences in isoelectric point and molecular weight, in which the protein is quantified by staining the protein with a Coomassie brilliant blue dye and quantifying each spot area and color strength (refer, for example, to J. Biol. Chem. 250: 4007-4021 (1975)).

Though the DNPH has been used as a reagent for detecting the carbonylation of a protein induced by oxidant stress, as described above, since the detection method has relied on the absorption of light having the ultraviolet wavelength or antibody detection using an anti-DNP antibody, no long discussion on the oxidized site has been made. Furthermore, since the detection method has been implemented by the spectrometry of the light having the ultraviolet wavelength, there was a limit in the detection sensitivity.

On the other hand, the two-dimensional electrophoresis entails a problem of linearity and there are many cases where the size of the spots is not necessarily proportional to the actual quantity. In addition, since the image analysis is a three-dimensional evaluation method in consideration of the thickness of an electrophoresis gel, it entails a problem of needing a lot of labor and time.

Under these circumstances, new methods of analysis for protein utilizing mass spectrometry have been proposed (refer, for example, JP-A 2005-345332 and JP-A 2005-315688). JP-A 2005-345332, for example, discloses a method of mass spectrometry comprising the steps of preparing samples containing plural analytes labeled with isotopic reagents having different molecular weights, separating the sample by chromatography, subjecting the separated samples to mass spectrometry using a tandem mass spectrometer capable of multi-dissociation measurement, analyzing results of the mass spectrometry in real time and utilizing results of the analysis of at least one analyte in real time for mass spectrometry of another analyte. In the invention described in JP-A 2005-345332, proteins or peptides obtained by decomposition of protein are cited as the analytes to make it possible to identify and quantify biopolymers with high precision and high throughput.

The invention described in JP-A 2005-315688 relates to detection and quantification of in vivo proteins damaged by oxidation, to a tag for analyzing the proteins damaged by oxidation, which is used for determining the in vivo sites damaged by oxidation, and to a method of analyzing the proteins damaged by oxidation using the tag. The method of analyzing the proteins damaged by oxidation disclosed in JP-A 2005-315688 comprises mixing both the tag for analysis and a tag for analysis labeled with an isotope with a sample, bringing the resultant mixture into contact with a carrier that retains metal atoms to be linked to an affinity peptide tag, recovering from the carrier a complex of the proteins damaged by oxidation and the tag for analysis and using the mass spectrometry to detect doublet peaks emerging due to a difference in molecular weight. The tag for analyzing the proteins damaged by oxidation comprises at least one residue having affinity peptide, a linker and a reaction group and has at least three (X-His) units, in which X stands for an arbitrary amino acid or an amino acid derivative.

Both the inventions described in JP-A 2005-345332 and JP-A 2005-315688 perform labeling with an isotope reagent and utilize a pair of peaks (doublet spectra) emerging in consequence of mass spectrometry to specify the proteins to be analyzed and are therefore at an advantage in attaining easier and more infallible specification than the case of merely performing labeling with a labeling reagent.

In the mass spectrometry of proteins, however, various peaks emerge and, since it cannot be denied that mere labeling with an isotopic reagent allows peak pairs to be possibly overlapped with other peaks, easy and infallible specification is not always attained.

In addition, the invention described in JP-A 2005-345332 neither refers to an analysis of a carbonylated protein that has undergone oxidative modifications nor concretely describes any isotopic reagent for labeling, for example. On the other hand, in the invention described in Patent Document 2, since the tag for analysis is required to include a linker etc., there is a fair possibility of the analysis precision being lowered because the composition synthesis of the tag is bothersome and because usage of linkers increases the fragmentions. Furthermore, in the invention described in JP-A 2005-315688, as described above, since the mixture has to be brought into contact with the carrier that retains metal atoms to be linked to the affinity peptide tag that is a marker for analysis, the operation thereof is complicated.

### DISCLOSURE OF THE INVENTION

The present invention has been proposed in view of the problems encountered by the related diagrams and one object thereof is to provide a method of quantitative analysis of oxidized proteins, which method can easily and infallibly specify peaks resulting from oxidized proteins that have been labeled in mass spectrometry and can rapidly quantify the oxidized proteins with high sensitivity. Another object of the present invention is to provide labeling reagents for quantitative analysis of oxidized proteins, which reagents are easy to synthesize and capable of being specifically linked to specific sites of the oxidized proteins (carbonylated proteins) to label the oxidized proteins and also provide a labeling reagent kit for quantitative analysis of the oxidized proteins.

The present invention provides a method of quantitative analysis of oxidized proteins, comprising the steps of labeling oxidized proteins, which have undergone oxidative modifications, with labeling reagents comprising a first labeling reagent capable of reacting with the oxidized proteins and a second labeling reagent having a same chemical structure as the first labeling reagent and having at least part of constituting atoms substituted by the isotopic atoms concerned; mixing the oxidized proteins labeled with the first labeling reagent and the oxidized proteins labeled with the second labeling reagent together to form mixtures; and subjecting the mixtures to mass spectrometric analysis with mixing ratios varied.

When an oxidized protein labeled with the first labeling reagent and an oxidized protein labeled with the second labeling reagent are mixed together to measure the mass spectrometry, the peak pairs having mass differences between the first and second labeling reagents emerge in the form of the spectra. This method, however, has a possibility that the peak pairs will be overlapped with other peaks to make it difficult to attain infallible discrimination. In the present invention, therefore, the mass spectra are observed showing the peak ratios of the oxidized proteins between labeling with the first labeling reagent and labeling with the second labeling reagent. For example, the opposite relationship in height between the peak pairs makes an appearance between the cases as follows; the case where the ratio of the oxidized protein labeled with the first labeling reagent is larger than that of the oxidized protein labeled with the second labeling reagent to perform the mass spectrometry and the case where the ratio of the oxidized protein labeled with the first labeling reagent is smaller than that of the oxidized protein labeled with the second labeling reagent to perform the mass spectrometry. The peaks resulting from the oxidized protein labeled utilizing this counterpart in height of the peak pairs are specified and quantified.

On the other hand, the labeling reagent for quantitative analysis of oxidized proteins according to the present invention contains 2,4-dinitrophenylhydrazine in which six carbon atoms on a phenyl group have been substituted by the carbon isotopes. Furthermore, the labeling reagent kit for quantitative analysis of oxidized proteins according to the present invention comprises a first labeling reagent containing 2,4-dinitrophenylhydrazine and a second reagent containing 2,4-dinitrophenylhydrazine in which the six carbon atoms on a phenyl group have been substituted by the carbon isotopes of the same number.

The 2,4-dinitrophenylhydrazine (DNPH) forms a stable Schiff's base in cooperation with an oxidized protein (carbonylated protein) that has undergone oxidative modifications due to oxidant stress, and the Schiff's base is linked to the carbonylated protein to quantify the resultant. The carbons on the phenyl group of the 2,4-dinitrophenylhydrazine are substituted by carbon isotopes (¹³C) to form 2,4-dinitrophenylhydrazine (¹³C₆-DNPH) different only in molecular mass by 6 from the DNPH. When the carbonylated protein is labeled with the ¹³C₆-DNPH, the resultant is separated by chromatography as the same peak as in the case where the carbonylated protein has been labeled with the DNPH. In view of this, when carbonylated proteins are labeled with both the DNPH and the ¹³C-DNPH, and separated by chromatography to be measured with the mass spectrometry, the peak pairs having a mass difference of 6 emerge in the form of spectra. Since the labeling reagent (¹³C₆-DNPH) for quantitative analysis of proteins according to the present invention does not need use of a linker, this method is easy to prepare the samples and does not lower the analysis precision.

According to the present invention, an oxidized protein (carbonylated protein) is capable of rapidly quantifying with high sensitivity. Therefore, the present invention is expected to enable provision of data useful for making a facile connection between the oxidation of a protein and disorders, for example. Furthermore, the invented quantification of an oxidized protein is promising for facility without a lot of labor and time and for attaining high reproducibility irrespective of skillfulness.

### BRIEF DESCRIPTION OF THE DRAWINGS

FIG. 1 is a diagram showing peak pairs emerging by labeling.

FIG. 2 shows reverse of peak intensities of peak pairs.

FIG. 3(a) is a schematic view showing induction of ¹²C-DNPH and FIG. 3(b) a schematic view showing induction of ¹³C-DNPH.

FIG. 4 is a diagram showing one example of a scheme of synthesizing ¹³C-DNPH.

FIG. 5 is a mass spectrometry chart showing samples having ¹²C-DNPH-induced oxidized myoglobin and ¹³C-DNPH-induced oxidized myoglobin subjected to enzymatic digestion when ¹²C-DNPH-induced protein:¹³C-DNPH-induced protein=100:30 (in molar ratio).

FIG. 6 is a mass spectrum showing peaks related to ¹²C-DNPH-induced oxidized myoglobin and ¹³C-DNPH-induced oxidized myoglobin subjected to enzymatic digestion when ¹²C-DNPH-induced protein: ¹³C-DNPH-induced protein=30:100 (in molar ratio).

FIG. 7 is a mass spectrum showing peaks related to ¹²C-DNPH-induced oxidized lysozyme and ¹³C-DNPH-induced oxidized lysozyme subjected to enzymatic digestion when (¹²C-DNPH-induced protein):(¹³C-DNPH-induced protein)=100:30 (in molar ratio).

FIG. 8 is a mass spectrum showing peaks related to ¹²C-DNPH-induced oxidized lysozyme and ¹³C-DNPH-induced oxidized lysozyme subjected to enzymatic digestion when (¹²C-DNPH-induced protein):(¹³C-DNPH-induced protein)=30:100 (in molar ratio).

### BEST MODE FOR CARRYING OUT THE INVENTION

A method of quantitative analysis of an oxidized protein, labeling reagents for quantitative analysis of the oxidized protein and a labeling reagent kit for quantitative analysis of the oxidized protein according to the present invention will be described hereinafter in detail.

In the method of quantitative analysis of an oxidized protein according to the present invention, an oxidized protein that has undergone oxidative modifications (a carbonylated protein, for example) is labeled with a labeling reagent to perform mass spectrometry. The protein undergoes oxidative modifications due to oxidant stress to oxidize part of amino acids to produce various oxidation products. The oxidation products of amino acids and mass changes by oxidation are shown in Table 1.

TABLE 1

| Amino Acids | Oxidation Products and Mass Differences |
|---|---|
| Cys | sulfonic acid (+48), sulfinic acid (+32), hydroxyl (+16) |
| Cystine | sulfonic acid (+48), sulfinic acid (+32) |
| Met | sulfoxide (+18), sulfone (+32), aldehyde (−32) |
| Trp | hydroxy (+16, +32, +48 . . . ), pyrol ring-open (+32) |
| Tyr | hydroxy (+16, +32) |
| Phe | hydroxy (+16, +32) |
| His | oxo- (+16), ring-open (−22, −10, +5) |
| Leu | hydroxy (+16), carbonyl (+14) |
| Ile | hydroxy (+16), carbonyl (+14) |
| Val | hydroxy (+16), carbonyl (+14) |
| Pro | hydroxy (+16), carbonyl (+14) |
| Arg | deguanidination (−43), hydroxy (+16), carbonyl (+14) |
| Lys | hydroxy (+16), carbonyl (+14) |
| Glu | decarboxylation (−30), hydroxy (+16), carbonyl (+14) |
| Asp | decarboxylation (−30), hydroxy (+16) |
| Gln | hydroxy (+16), carbonyl (+14) |
| Asn | hydroxy (+16) |
| Ser | hydroxy (+16), carbonyl (−2, +O—H₂O) |
| Thr | hydroxy (+16), carbonyl (−2, +O—H₂O) |
| Ala | hydroxy (+16) |

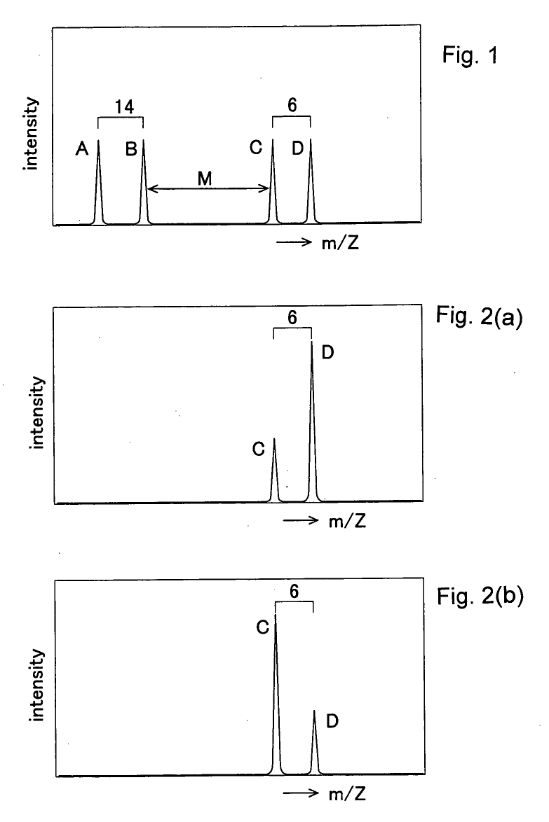

When the proteins have undergone oxidative modifications, they are changed in mass as shown in Table 1 depending on oxidation products of amino acids. When an amino acid has been carbonylated, for example, a mass difference is 14. Therefore, as a result of mass spectrometry, as shown in FIG. 1, a peak B resulting from the oxidized protein (carbonylated protein) emerges at a position of +14 relative to a peak A resulting from the protein having been subjected to the mass spectrometry.

As shown in Table 1, the proteins are formed with various functional groups (carbonyl group, for example) by oxidation. When a labeling reagent reacting with the functional group is allowed to act on the proteins, the labeling reagent is linked thereto to form a labeled protein. A mass difference (molecular mass difference) M between the labeled protein and the oxidized protein not labeled is obtained by subtracting the molecular mass lost by the reaction from the molecular mass of the labeling reagent and, as shown in FIG. 1, a peak C resulting from the labeled protein emerges at a position of +M relative to the peak B resulting from the oxidized protein.

In the method of the present invention, labeling is performed using a first labeling reagent reacting with an oxidized protein and a second labeling reagent having the same chemical structure as the first labeling reagent and having at least part of component, atoms substituted by isotopes of the relevant atoms. Since the second labeling reagent has the same chemical structure as the first labeling reagent and has at least part of the component atoms substituted by the isotopes of the relevant atoms, the molecular mass of the second labeling agent differs by the number of the isotopes having substituted the atoms from the molecular mass of the first labeling reagent. Assuming that six carbon atoms on a phenyl group of the second labeling reagent are substituted by stable isotopes ¹³C, for example, the mass difference between the first and second labeling reagents becomes 6. When the oxidized proteins are labeled with the first and second labeling reagents, a first labeled protein labeled with the first labeling reagent and a second labeled protein labeled with the second labeling reagent are produced. In the mass spectrum, therefore, the peak C that results from the first labeled protein and a peak D that results from the second labeled protein and from the peak C emerge. The mass difference between the peaks C and D is 6.

Use of the first and second labeling reagents allows a peak pair (peaks C and D) to emerge, and structure of the peak pair results in structure of the peak resulting from the oxidized protein. In the mass spectrometry of proteins, however, a great number of peaks emerge in major possibilities. Therefore, there is a case where the peak pair mingles in the great number of peaks to be difficult to correctly discriminate.

In the present invention, therefore, the labeling is performed, with the ratio between the first and second labeling agents varied, and the mass spectrometry is performed in the individual cases to enable the peak pair to be infallibly confirmed. To be specific, labeling is first performed, with the ratio of the first labeling reagent made smaller than that of the second labeling reagent, and mass spectrometry is performed. As a result, as shown in FIG. 2(a), the peak D resulting from the second labeled protein labeled with the second labeling reagent is larger in height (peak intensity) than the peak C resulting from the first labeled protein labeled with the first labeling reagent. Next, the labeling is performed, with the ratio of the first labeling reagent made larger than that of the second labeling reagent, and mass spectrometry is performed. As a result, as shown in FIG. 2(b), the peak D resulting from the second labeled protein labeled with the second labeling reagent is smaller in height (peak intensity) than the peak C resulting from the first labeled protein labeled with the first labeling reagent.

Therefore, by performing the labeling and mass spectrometry twice, the peak pairs having the heights reversed can be specified as the peak pair resulting from the oxidized protein. When the peak pairs having the heights of the peaks reversed can be confirmed, the target peak pair can be discriminated even in the presence of a great number of peaks. As a method of performing labeling, with the ratio between the first and second labeling reagents varied, a method comprising labeling oxidized proteins with the first and second labeling reagents, respectively, and mixing the labeled oxidized proteins at different ratios can be adopted, for example. Though the case, in which the labeling and mass spectrometry processes are performed twice, has been described here, the labeling process with the first and second labeling reagents at different ratios and the mass spectroscopy process may be performed three times or more.

The mass spectrometry can be performed without subjecting the labeled analysis samples (labeled proteins) to any pretreatment. It can be performed, when the molecular weights of the proteins are very large, after subjecting the proteins to enzymatic digestion to disintegrate the resultant proteins into components having relatively small molecular weights. In consequence of the disintegration, there are cases where peak pairs of plural components are observed and where the target peak pair can be confirmed more clearly.

Also, in the mass spectrometry, tandem mass spectrometry (MS/MS measurement) is performed as regards peaks resulting from the oxidized proteins to enable analysis of the oxidized sites of the oxidized proteins. In the MS/MS measurement, all ions produced from an ion source are separated using a first mass spectrometer, and only a target pair is subjected to selective fragmentation to perform analysis thereof with a second mass spectrometer. In peptide MS/MS measurement, an amino-acid sequence can be analyzed.

Next, the labeling reagents used in the method of quantitative analysis of the oxidized proteins will be described. As described in the foregoing, the method of quantitative analysis of the oxidized proteins according to the present invention needs use of the labeling reagents for labeling the oxidized proteins. Here, the labeling reagents are required to react with the oxidized sites of the oxidized proteins and to be selected in accordance with the oxidation products of the amino acids shown in Table 1. In the present invention, since a carbonylated protein that undergoes oxidative modifications is the target measurement object, 2,4-dinitrophenylhydrazine (DNPH) represented by chemical formula 1 is used as the labeling reagent.

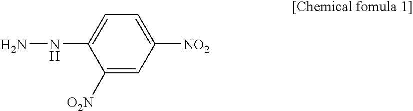

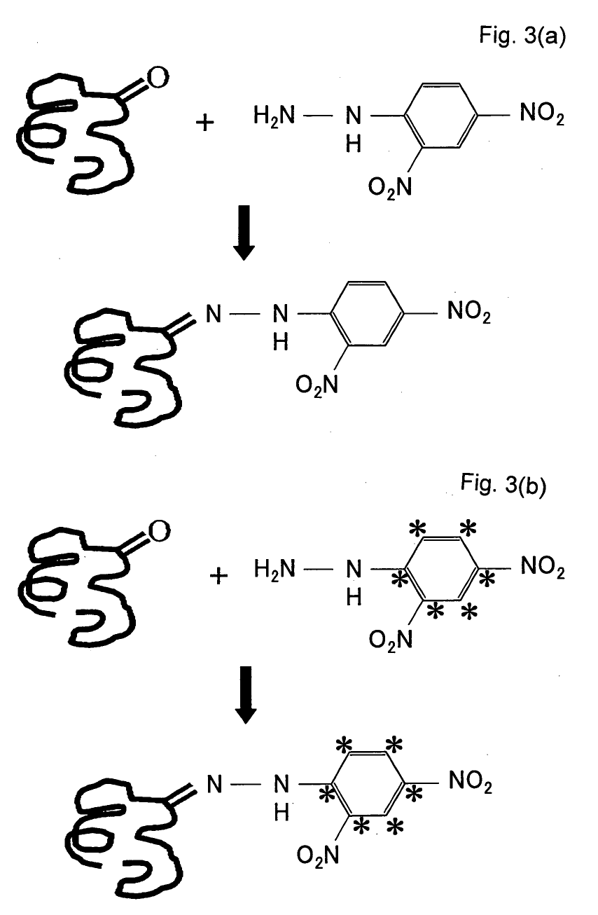

The DNPH reacts with a carbonyl group of an oxidized protein (carbonylated protein) as shown in FIG. 3(a) to form a stable Schiff's base, thereby forming a DNPH-induced protein. The mass number difference between the DNPH-induced protein and the carbonylated protein is expressed by (the mass number (198) of DNPH)−(the mass number (18) of H₂O)=180, and that between the DNPH-induced protein and the protein before being carbonylated is 194. The DNPH is used as the first labeling reagent and, at the same time, 2,4-dinitrophenylhydrazine (¹³C-DNPH) having six carbon atoms on the phenyl group of the DNPH substituted by stable carbon isotopes (¹³C) is used as the second labeling reagent.

As shown in FIG. 3(b), the ¹³C-DNPH reacts with the carbonyl group of the carbonylated protein, similarly to the DNPH, to produce a ¹³C-DNPH-induced protein. The ¹³C-DNPH has a larger mass number by 6 than the DNPH, and the difference in mass number between the ¹³C-DNPH-induced protein and the DNPH-induced protein is 6. Therefore, by mixing the carbonylated protein labeled with the DNPH and the carbonylated protein labeled with the ¹³C-DNPH and subjecting the resultant mixture to mass spectrometry, a peak pair having a difference of 6 in mass number can be observed. Incidentally, in identifying the carbonylated protein through the mass spectrometry, a method comprising segmenting the carbonylated protein with a protease, such as trypsin, into small peptides and measuring the peptides is generally adopted.

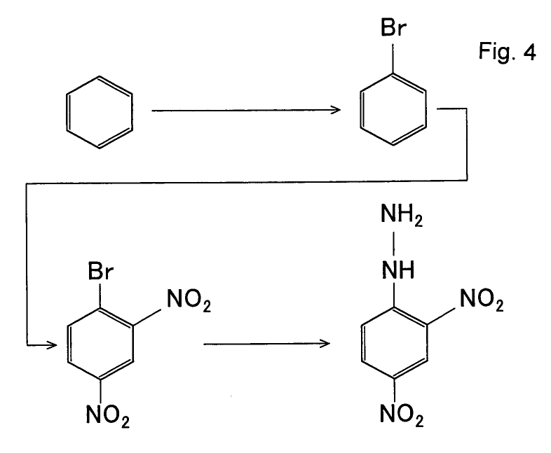

The ¹³C-DNPH has been synthesized for the first time by the present inventors and can be synthesized by the law of the art, with benzene having six carbon atoms substituted by stable isotopes (¹³C) as the starting substance. FIG. 4 shows a scheme of synthesizing ¹³C-DNPH. In order to synthesize the ¹³C-DNPH, benzene is brominated to form bromobenzene, and a nitro group is introduced into the bromobenzene to obtain 2,4-dinitrobromobenzene. Hydrazine hydrate is allowed to react on the 2,4-dinitrobromobenzene thus synthesized to obtain 2,4-dinitrophenylhydrazine. Use of benzene having six carbon atoms substituted by stable isotopes (¹³C) as the starting substance enables 2,4-dinitrophenylhydrazine (¹³C-DNPH) having six carbon atoms on the phenyl group substituted by stable isotopes (¹³C) to be synthesized.

### Example

A concrete example of the present invention will be described based on experimental results.

### Synthesis of ¹³C₆-DNPH

To 0.441 g (65 mmol) of benzene (¹³C₆) having six carbon atoms substituted by stable isotopes (¹³C), 1 mg of Fe and 13 ml (5.29 mmol) of Br₂ were added and the resultant was stirred at 55° C. for 15 min. The resultant mixture was cooled to room temperature, an aqueous 10% sodium hydroxide solution was added to the cooled mixture, and extraction was performed with diethyl ether. The resultant was washed with water and then distilled to obtain bromobenzene.

Next, 7.5 ml of sulfuric acid (H₂SO₄) and 5.0 ml of nitric acid were mixed and heated to 85° C. while being stirred and were then added with the bromobenzene has already been synthesized. The resultant mixture was further stirred at 85° C. and then cooled to room temperature. The resultant was then cooled with ice and extraction is performed using diethyl ether. The extracted substance was washed with water, then evaporated under reduced pressure, and developed using a thin-layer chromatography to determine separation conditions for the target compound. The target compound was refined through a silica gel column, and the product obtained was analyzed with a ¹H-NMR. As a result, the production of dinitrobromobenzene was confirmed. The yield of the dinitrobromobenzene was 316.3 mg (2.25 mmol), and the overall yield from benzene (¹³C₆) was 22.1%.

Subsequently, the dinitrobromobenzene was added with 4.0 ml of ethanol, and the resultant mixture was further added with 0.40 ml of hydrazine hydrate and 1.50 ml of ethanol while it was being stirred at 65° C. After continuing stirring at 65° C., the resultant was cooled to room temperature and filtered. The filtered substance was washed with cooled ethanol and dried under reduced pressure to obtain ¹³C₆-DNPH. The yield of the ¹³C₆-DNPH obtained was 201.0 mg (0.99 mmol), and the overall yield from the raw material benzene (¹³C₆) was 17.4%.

Labeling and Mass Spectrometry of Myoglobin:

(Labeling with ¹²C-DNPH)

To 100 mL of 0.1 mM protein (myoglobin), 100 mM PBS (pH: 7.4) and 2 mL of NaOCl (diluted to 1/100) were added to make a reaction at −85° C. for 30 min. As a result, the protein was oxidized (carbonylated).

Next, the reactant was precipitated using acetone and added successively with reagents shown below, and the resultant mixture was allowed to react in a dark room at room temperature for 30 min. As a result, ¹²C-DNPH-induced, carbonylated myoglobin was obtained.

10 mM Tris-HCl (pH: 8.0): 50 μL

10% SDS: 5 μL

10 mM ¹²C-DNPH/2N—HCl: 50 μL

(Labeling with ¹³C-DNPH)

To 30 mL (3 nmol) of 0.1 mM protein (myoglobin), 100 mM PBS (pH: 7.4) and 2 nL of NaOCl (diluted to 1/100) were added to make a reaction at −85° C. for 30 min. As a result, the protein was oxidized (carbonylated).

Next, the reactant was precipitated using acetone, added successively with the reagents shown below, and reacted in a dark room at room temperature for 30 min. As a consequence, ¹³C-DNPH-induced, carbonylated myoblobin was obtained.

10 mM Tris-HCl (pH: 8.0): 50 μL

10% SDS: 5 μL

10 mM ¹³C-DNPH/2N—HCl: 50 μL

(Mixing of ¹²C-DNPH-induced proteins and ¹³C-DNPH-induced proteins and analysis of the resultant mixture)

The ¹²C-DNPH-induced proteins and ¹³C-DNPH-induced proteins thus synthesized were mixed (¹²C-DNPH-induced proteins ¹³C-DNPH-induced proteins=100:30), precipitated using acetone and added with 50 μL of 6 M urea/25 mM ammonium hydrogencarbonate and 2 μL of 200 mM DTT to make a reaction at 37° C. for one hour. Then, 450 μL of 25 mM ammonium hydrogencarbonate and trypsin were added to the resultant mixture. The subsequent mixture was subjected to enzymatic digestion at 37° C. around the clock. The resultant was evaporated under reduced pressure to 10 μL to 20 μL, and 0.5 μL thereof was subjected to mass spectrometry using ZipTipC18 (elut in 10 μL). As a matrix solvent, CHCA/50% acetonitrile was used. Furthermore, the mass spectrometry was performed using an MALDI-TOF/MS (Matrix-Assisted Laser Desorption/Ionization Time-Of-Flight Mass Spectrometer).

### (Mass Spectrometry Results)

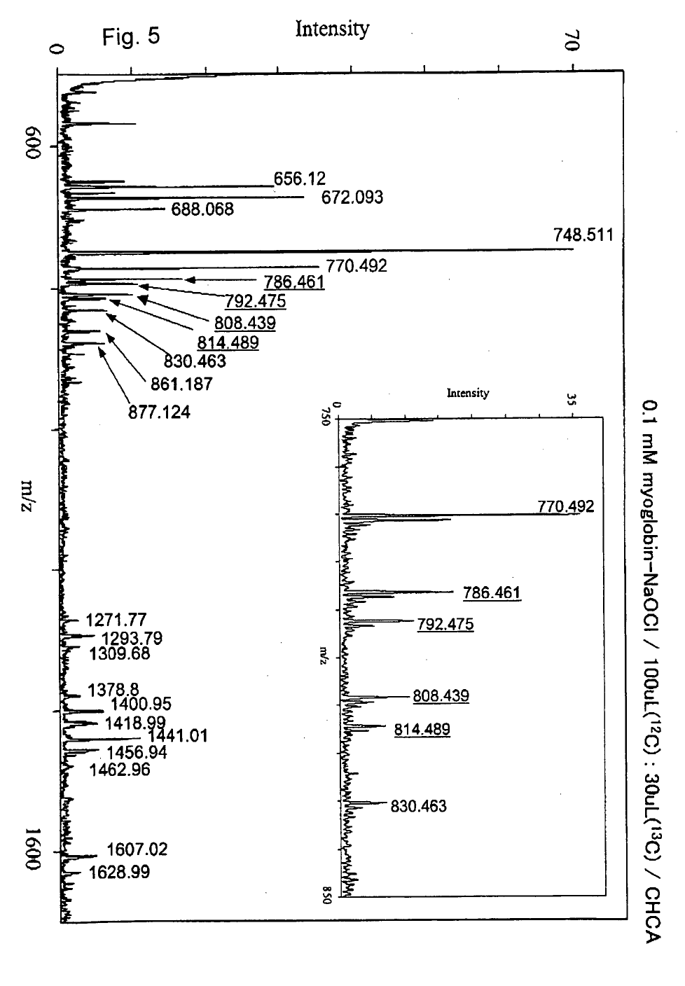

FIG. 5 shows a chart of mass spectrometry by the MALDI-TOF/MS. In FIG. 5, two peak pairs, one having a peak of mass of 786.461 and a peak of mass of 792.475 and the other having a peak of mass of 808.439 and a peak of mass of 814.489, were observed as the peak pairs having a difference of 6 in atomic mass number. Furthermore, in the respective peak pairs, the peaks resulting from ¹²C-DNPH-induced proteins (the peak of mass of 786.461 and the peak of mass of 808.439) have larger peak intensities.

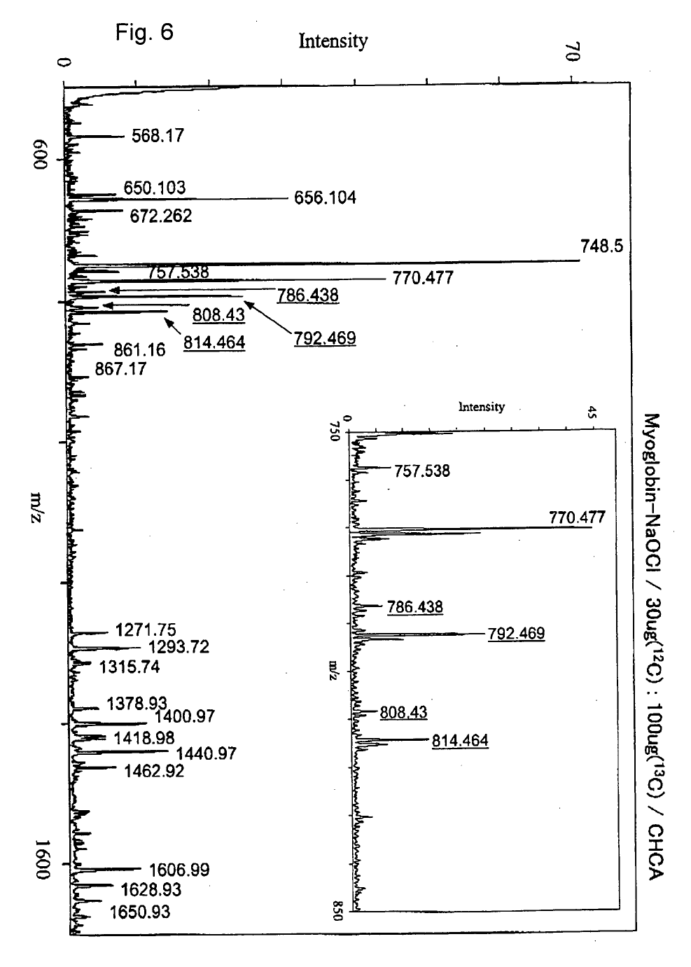

Subsequently, therefore, labeling with ¹²C-DNPH, labeling with ¹³C-DNPH, mixing of ¹²C-DNPH-induced proteins and ¹³C-DNPH-induced proteins and mass spectrometry were performed in same manner as described above except that adjustment was made so that ¹²C-DNPH-induced proteins:¹³C-DNPH-induced proteins=30:100 (in molar ratio). FIG. 6 shows a chart of mass spectrometry in this case.

Also in this case, two peak pairs, one having a peak of mass of 786.461 and a peak of mass of 792.475 and the other having a peak of mass of 808.439 and a peak of mass of 814.489, were observed as the peak pairs having a difference of 6 in atomic mass number. In the respective cases, however, the peaks resulting from the ¹²C-DNPH-induced proteins (the peak of mass of 786.461 and the peak of 808.439) have lower peak intensities.

It could be concluded from these facts that the two peak pairs resulted from the myoglobin that underwent oxidative modifications. In addition, these peaks were subjected to MS/MS measurement to enable analysis of the oxidized sites of myoglobin.

Labeling and Mass Spectrometry of Oxidized Lysozyme

Oxidation was performed by following the procedure in the case of the myoglobin, and labeling with ¹²C-DNPH and labeling with ¹³C-DNPH were performed. The labeled proteins were mixed and subjected mass spectrometry. The method of oxidation, the method of labeling, the mixing and the mass spectrometry were performed pursuant to the case of the myoglobin.

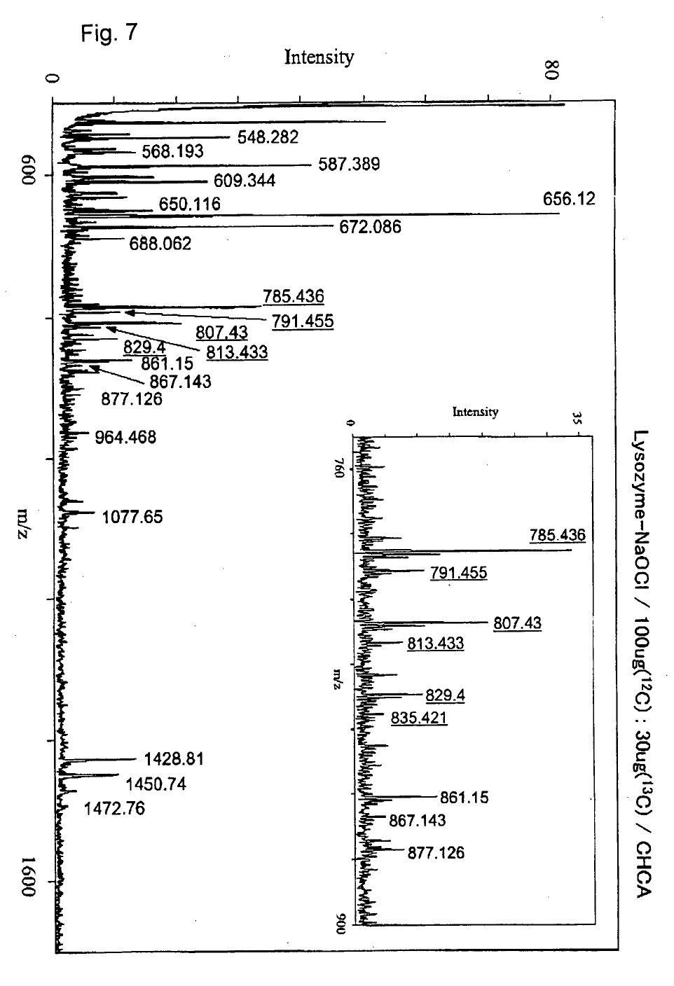

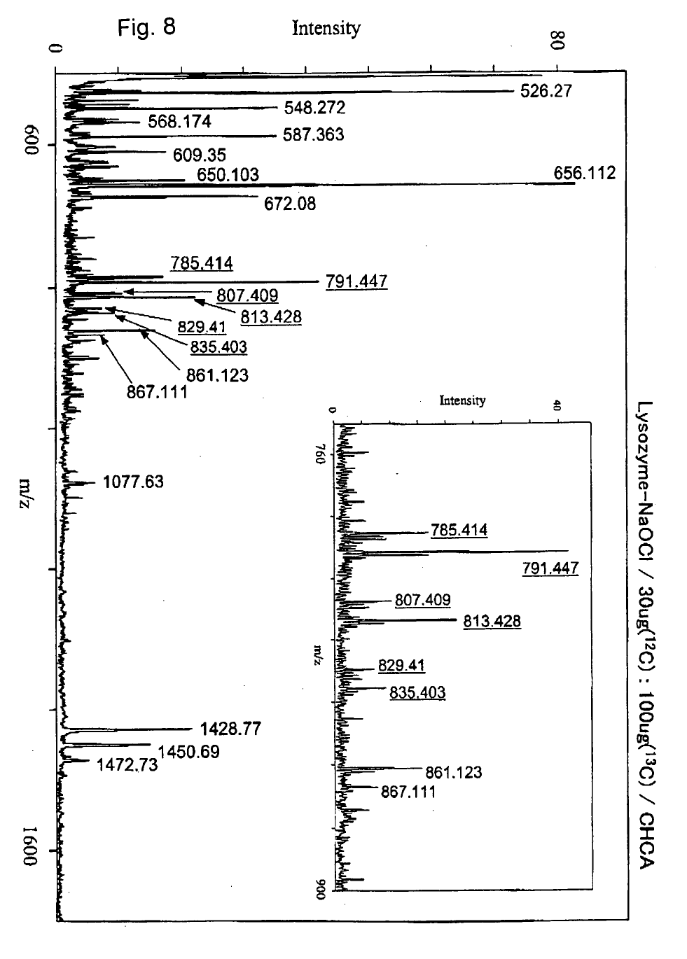

FIG. 7 shows a mass spectrometry chart in the case where ¹²C-DNPH-induced proteins:¹³C-DNPH-induced proteins=100:30, and FIG. 8 shows a mass spectrometry chart in the case where ¹²C-DNPH-induced proteins:¹³C-DNPH-induced proteins=30:100 (in molar ratio). Three peak pairs, one having a peak of mass of 785.436 and a peak of mass of 791.455, another having a peak of mass of 807.43 and a peak of mass of 813.433 and the remaining one having a peak of mass of 829.4 and a peak of mass of 835.421 were observed as the peak pairs having a difference of 6 in atomic mass number. Furthermore, since both the mass spectrometry chart shown in FIG. 7 and the mass spectrometry chart shown in FIG. 8 are reversed, it was confirmed that the peak pairs were resulted from the oxidized lysozyme.

1. A method of quantitative analysis of oxidized proteins, comprising:
labeling oxidized proteins, which have undergone oxidative modifications, with labeling reagents comprising a first labeling reagent capable of reacting with the oxidized proteins and a second labeling reagent having a same chemical structure as the first reagent and having at least part of component atoms substituted by an isotope of an atom concerned;
mixing the oxidized proteins labeled with the first labeling reagent and the oxidized proteins labeled with the second labeling reagent together to form mixtures; and
subjecting the mixtures to mass spectrometry, with mixing ratios varied.

2. A method of quantitative analysis of oxidized proteins according to claim 1, wherein the second reagent has carbon atoms as the component atoms, part of which is substituted by a carbon isotope.

3. A method of quantitative analysis of oxidized proteins according to claim 2, wherein the second reagent includes a benzene ring having six carbon atoms substituted by carbon isotopes, and the first and second reagents have a difference of 6 in molecular weight between them.

4. A method of quantitative analysis of oxidized proteins according to claim 1, wherein the oxidized proteins comprise carbonylated proteins.

5. A method of quantitative analysis of oxidized proteins according to claim 4, wherein the first labeling reagent comprises 2,4-dinitrophenylhydrazine, and the second reagent comprises 2,4-dinitrophenylhydrazine in which six carbon atoms on a phenyl group have been substituted by carbon isotopes.

6. A method of quantitative analysis of oxidized proteins according to claim 1, further comprising subjecting an analysis sample to enzymatic digestion before the mass spectrometry.

7. A method of quantitative analysis of oxidized proteins according to claim 1, the mass spectrometry is tandem mass spectrometry to analyze oxidized site of the oxidized proteins.

8. A labeling reagent for quantitative analysis of oxidized proteins, containing 2,4,-dinitrophenylhydrazine in which six carbon atoms on a phenyl group have been substituted by carbon isotopes.

9. A labeling reagent kit for quantitative analysis of oxidized proteins, comprising a first labeling reagent containing 2,4-dinitrophenylhydrazine and a second reagent containing 2,4,-dinitrophenylhydrazine in which six carbon atoms on a phenyl group have been substituted by carbon isotopes.
